(19) United States
(12) Patent Application Publication
(10) Pub. No.: US 2024/0366700 A1
(43) Pub. Date: Nov. 7, 2024

(54) HERBAL MALE ENHANCEMENT AND ENERGY FORMULATION

(71) Applicant: Robert R. Beltran, Rancho Santa Fe, CA (US)

(72) Inventor: Robert R. Beltran, Rancho Santa Fe, CA (US)

(21) Appl. No.: 18/142,301

(22) Filed: May 2, 2023

(51) Int. Cl. A61K 36/185; A61K 31/198; A61K 31/205; A61K 31/355; A61K 33/30; A61K 36/13; A61K 36/15; A61K 36/258; A61K 36/31; A61K 36/42; A61K 36/45; A61K 36/48; A61K 36/58; A61K 36/67; A61K 36/71; A61K 36/734; A61K 36/77; A61K 36/889; A61K 36/899; A61K 36/90

(52) U.S. Cl. CPC A61K 36/185; A61K 31/198; A61K 31/205; A61K 31/355; A61K 33/30; A61K 36/13; A61K 36/15; A61K 36/258; A61K 36/31; A61K 36/42; A61K 36/45; A61K 36/48; A61K 36/58; A61K 36/67; A61K 36/71; A61K 36/734; A61K 36/77; A61K 36/889; A61K 36/899; A61K 36/90

(57) ABSTRACT

The present disclosure relates to an herbal composition and a method of administering the composition including administering the composition in pill or capsule form on a routine basis for the purpose of increasing energy, alertness, clarity, memory, and sexual enhancement in individuals, and particularly in males.

### TECHNICAL FIELD

The embodiments generally relate to the field of herbal formulations and methods of administering herbal formulations.

### BACKGROUND

Formulations and methods of administering formulations designed for increased energy, alertness, mental capacity, and sexual enhancement may include a variety of undesirable side effects such as shakiness, jitters, lethargy, nausea, high blood pressure, low blood pressure, headache, upset stomach, back pain, muscle pain, stuffy nose, flushing, or dizziness and the like. Additionally, formulations designed for increased energy, alertness, mental capacity, and sexual enhancement The present disclosure relates to a composition of an herbal enhancement formulation and a method of administering said composition that may enhance energy while reducing undesirable side effects.

### SUMMARY OF THE INVENTION

This summary is provided to introduce a variety of concepts in a simplified form that is further disclosed in the detailed description of the embodiments. This summary is not intended to identify key or essential inventive concepts of the claimed subject matter, nor is it intended for determining the scope of the claimed subject matter.

The present disclosure relates to a safe and non-addictive composition including herbal extracts from roots, barks, berries, and leaves.

The present disclosure relates to a composition containing a number of components including, but not limited to, saw palmetto 8:1 extract; damiana leaf 8:1 extract; piper nigrum fruit 8:1 extract; hawthorn berry 8:1 extract; muira pauma bark 4:1 extract; Korean red ginseng 8:1 extract; catuaba bark 4:1 extract; epimedium sagittatum 8:1 extract; Ginkgo biloba leaf 8:1 extract; cuscata leaf 4:1 extract; tribulus terrestris vine 8:1 extract; butea superba root 8:1 extract; maca root 8:1 extract; pine bark extract 8:1 extract; cranberry extract; Avena sativa 8:1 extract; sarsaparilla root 8:1 extract; swedish flower pollen; pumpkin seed; di-tocopheral acetate; zinc aspartate 20% elemental; L-carnitine; L-arginine HCl; L-lysine; and Tong-Kat Ali 200:1.

Both the foregoing general description and the following detailed description present embodiments intended to provide an overview or framework for understanding the nature and character of the embodiments disclosed herein. Any accompanying drawings are included to provide further understanding and are incorporated into and constitute a part of this specification. The drawings illustrate various embodiments of the disclosure, and together with the description explain the principles and operations thereof.

### BRIEF DESCRIPTION OF THE DRAWINGS

The features and advantages of the present disclosure will be more fully described in, or rendered obvious by the following detailed description of the preferred embodiments, which are to be considered together with the accompanying drawings wherein like numbers refer to like parts and further, wherein:

FIG. 1 depicts observed enhancement in key areas in participants receiving administration of the method as described herein.

The drawings are not necessarily to scale, and certain features and certain views of the drawings may be shown exaggerated in scale or in schematic in the interest of clarity and conciseness.

### DETAILED DESCRIPTION

The specific details of the single embodiment or variety of embodiments described herein are to the described system or methods of use. Any specific details of the embodiments are used for demonstration purposes only and no unnecessary limitations or inferences are to be understood from there.

The specific details of single embodiments or variations described herein are to the described composition and methods of use. Any specific details of the embodiments are used for demonstration purposes only and no unnecessary limitations or inferences are to be understood from there.

The present disclosure discloses a composition containing a number of components including, but not limited to, saw palmetto 8:1 extract; damiana leaf 8:1 extract; piper nigrum fruit 8:1 extract; hawthorn berry 8:1 extract; muira pauma bark 4:1 extract; Korean red ginseng 8:1 extract; catuaba bark 4:1 extract; epimedium sagittatum 8:1 extract; Ginkgo biloba leaf 8:1 extract; cuscata leaf 4:1 extract; tribulus terrestris vine 8:1 extract; butea superba root 8:1 extract; maca root 8:1 extract; pine bark extract 8:1 extract; cranberry extract; Avena sativa 8:1 extract; sarsaparilla root 8:1 extract; swedish flower pollen; pumpkin seed; di-tocopheral acetate; zinc aspartate 20% elemental; L-carnitine; L-arginine HCl; L-lysine; and Tong-Kat Ali 200:1.

According to some embodiments, the composition may further include magnesium stearate, silicon dioxide, or rice flour.

The compositions disclosed may be encapsulated in a pill or capsule form according to conventional means. According to some embodiments, the composition is ingested by a user at least once a day and in some embodiments, the composition is ingested up to four times daily. The composition, as best seen below in Table 1 may increase blood flow to the genital area including increased blood flow into the corpus cavernosum to produce increased erectile strength induration in addition to healthier tissue. The composition may have a direct effect on the nerve endings of the genital area to increase sensitivity. The composition may increase free testosterone in the bloodstream and may increase blood flow to the brain including areas of the brain active during sexual activity and facilitate the increase the release of pertinent neurotransmitters such as oxytocin, serotonin, and dopamine. The composition may have a direct effect on the pleasure areas of the brain and then in this manner may directly stimulate the release of neurotransmitters related to sexual activity. The composition may have a direct effect on microcirculation of blood to the brain and genital areas in addition to neurogenesis, such as the restoration and/or replacement of old or damaged neurons and brain matter.

According to various embodiments illustrating the practice of the present method and composition, a person having ordinary skill in the art will understand that the method and composition may be practiced or applied according to other variant embodiments. Many other possible modifications and variations of the details of the present composition and method based on different outlooks and applications may be made without departing from the spirit of this disclosure.

As best shown in Table 1, the composition may include various ingredients in a range of effective amounts according to the disclosed method.

TABLE 1

| | | 4 Capsule | |
|---|---|---|---|
| | Formulation 8:1 | Daily | |
| Component | Per Capsule | serving | |
| Saw Palmetto extract: 8:1 | 12.4 | 49.6 | mg |
| Damiana Leaf extract: 8:1 | 12.4 | 49.6 | mg |
| Piper Nigrum Fruit extract: | 5 | 20 | mg |
| Hawthorn Berry: 8:1 | 14.6 | 58.4 | mg |
| Muira Puama bark 4:1 extract: | 62 | 248 | mg |
| Korean Red Ginseng: 8:1 | 15 | 60 | mg |
| Catuaba bark 4:1 extract. | 90 | 360 | mg |
| Epimedium Saggitatum leaf: 8:1 | 6.6 | 26.4 | mg |
| Ginkgo Biloba leaf: 8:1 | 14 | 56 | mg |
| Cuscuta 4:1 leaf extract: | 20 | 80 | mg |
| Tribulis Terrestris vine: 8:1 | 12.2 | 48.8 | mg |
| Butea Superba root: 8:1 | 16.6 | 66.4 | mg |
| Maca root: 8:1 | 21.5 | 86 | mg |
| Pine Bark extract: 8:1 | 10.6 | 42.4 | mg |
| Cranberry extract: | 16 | 64 | mg |
| Avena Sativa extract seed: 8:1 | 2 | 8 | mg |
| Sarsaparilla root: 8:1 | 2 | 8 | mg |
| Swedish Flower (pollen): | 12.6 | 50.4 | mg |
| Pumpkin Seeds: | 7.6 | 30.4 | mg |
| Vitamin E (di-tocopherol acetate): | 2 | 8 | mg |
| Zinc Aspartate 20% elemental | 37.5 | 150 | mg |
| L-Carnitine: | 100 | 400 | mg |
| L-Arginine HCL: | 76 | 304 | mg |
| L-Lysine: | 60 | 240 | mg |
| TKA200 Tong-Kat Ali Root | 87.5 | 350 | mg |
| 200:1 Extract | | | |
| (1% Eurycomannones) | | | |

According to an example study of a method of administering the disclosed composition, participants ingested the disclosed composition once daily in the amount shown in Table 1. No participants were removed from the study due to medical issues including side effects. Participants reported multiple male climaxes without a refractory period. Study participants documented 5-15 male climaxes without a refractory period in a 30-45-minute sexual encounter. Participants reported duration and intensity of the male climax as significantly greater as per comparative analysis with competing herbal products. Participants reports higher number of climaxes, up to 15 in some cases, without a refractory period. This finding is not previously described in medical, lay public and competitor literature.

According to some embodiments, ingestion of the composition may occur two times daily wherein the composition includes the components in the amount listed in Table1. Participants reported increased energy including increased energy to a significant degree. One participant reported increased energy to a profound degree. All participants indicated that improved energy included no increased nervousness, anxiety, or jitteriness. All participants indicated significant improvement in mental clarity and acuity, mental alertness, well-being, and memory. Two participants reported significant improvement in general well-being and contentment, and two participants reported profound improvement in general well-being and contentment.

Sexual enhancement was measured according to numerous variables including anticipation prior to sexual activity, erectile strength or rigidity, pleasure or enjoyment during sexual activity including up to and just prior to climax or orgasm, intensity of each orgasm, duration of each orgasm, number of orgasms having a refractory period thereafter, estimations on refractory periods, number of orgasms without a subsequent refractory period, and time between orgasms.

Prior to the study of a method of administering the disclosed composition, participants reported their current sexual situation as “good to very good.” Prior to onset of the study, participants one and two reported sexual encounters two to three times per week participant three listed three to four sexual encounters per week. Participant four reported four to five sexual encounters per week.

After the initial three-month period described previously, all participants noted “mild but definite” improvement in the categories of anticipation prior to sexual activity, erectile strength or rigidity, pleasure or enjoyment during sexual activity including up to and just prior to climax or orgasm, intensity of each orgasm, and duration of each orgasm.

After the ingestion and twice daily administration of the composition as shown in Table 1 participants reported “good to very good” improvement in the categories of anticipation prior to sexual activity, erectile strength or rigidity, pleasure or enjoyment during sexual activity including up to and just prior to climax or orgasm, intensity of each orgasm, and duration of each orgasm.

After participants increased the ingestion of the disclosed composition to three times daily in the amount shown in Table 1, all participants reported “very good to excellent” improvement in the categories of anticipation prior to sexual activity, erectile strength or rigidity, pleasure or enjoyment during sexual activity including up to and just prior to climax or orgasm, intensity of each orgasm, and duration of each orgasm. Two participants reported an increase in number of orgasms having a refractory period thereafter.

After participants increased the ingestion of the disclosed composition to two times daily in the amount shown in Table 1, all participants reported “excellent to profound” improvement in in the categories of anticipation prior to sexual activity, erectile strength or rigidity, pleasure or enjoyment during sexual activity including up to and just prior to climax or orgasm, intensity of each orgasm, duration of each orgasm, and number of orgasms having a refractory period thereafter.

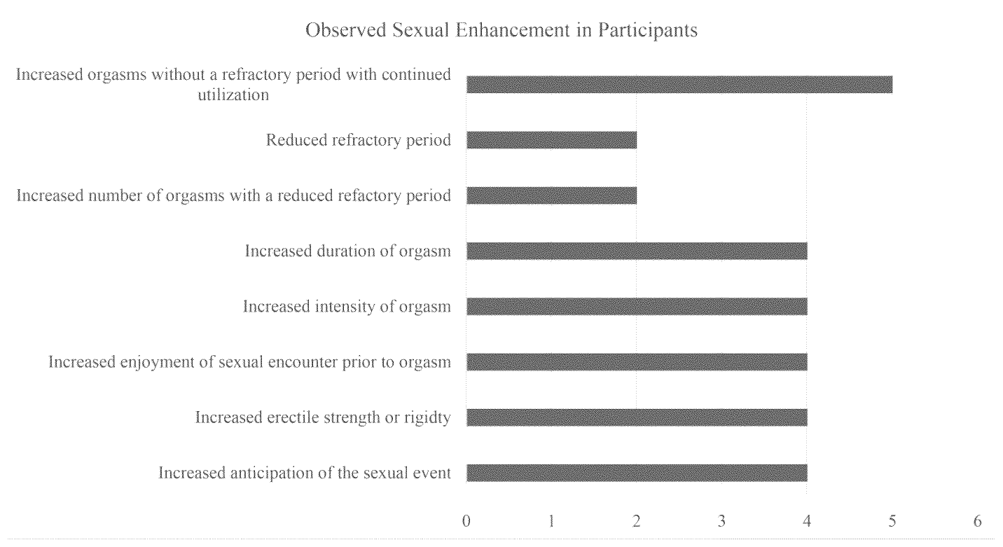

FIG. 1 depicts the previously described results including reported sexual enhancement for all participants in the categories of anticipation prior to sexual activity, erectile strength or rigidity, pleasure or enjoyment during sexual activity including up to and just prior to climax or orgasm, intensity of each orgasm, duration of each orgasm, number of orgasms having a refractory period thereafter, estimations on refractory periods, number of orgasms without a subsequent refractory period, and time between orgasms. According to some embodiments, with intake of maximum dosage and continued daily intake, study participants experienced multiple, in some cases 5-15, climaxes/orgasms without a refractory period in a single 30-45 minute encounter.

The following description of variants is only illustrative of components, elements, acts, product, and methods considered to be within the scope of the invention and are not in any way intended to limit such scope by what is specifically disclosed or not expressly set forth. The components, elements, acts, product, and methods as described herein may be combined and rearranged other than as expressly described herein and still are considered to be within the scope of the invention.

According to variation 1, a composition for sexual enhancement may include saw palmetto 8:1 extract; damiana leaf 8:1 extract; piper nigrum fruit 8:1 extract; hawthorn berry 8:1 extract; muira pauma bark 4:1 extract; Korean red ginseng 8:1 extract; catuaba bark 4:1 extract; epimedium sagittatum 8:1 extract; Ginkgo biloba leaf 8:1 extract; cuscata leaf 4:1 extract; tribulis terrestris vine 8:1 extract; butea superba root 8:1 extract; maca root 8:1 extract; pine bark extract 8:1 extract; cranberry extract; Avena sativa 8:1 extract; sarsaparilla root 8:1 extract; swedish flower pollen; pumpkin seed; di-tocopheral acetate; zinc aspartate 20% elemental; L-carnitine; L-arginine HCl; L-lysine; and Tong-Kat Ali 200:1.

Variation 2 may include composition for sexual enhancement as in variation 1, wherein the composition is encapsulated within one or more pills or capsules.

Variation 3 may include composition for sexual enhancement as in variation 1 or 2, wherein the composition includes saw palmetto 8:1 extract ranging from about 11.1-13.6 mg; damiana leaf 8:1 extract ranging from about 11.1-13.6 mg; piper nigrum fruit 8:1 extract ranging from about 4.5-5.5 mg; hawthorn berry 8:1 extract ranging from about 13.1-16.5 mg; muira pauma bark 4:1 extract ranging from about 56-68 mg; Korean red ginseng 8:1 extract ranging from about 13.5-16.5 mg; catuaba bark 4:1 extract ranging from about 81-99 mg; epimedium sagittatum 8:1 extract ranging from about 5.9-7.3 mg; Ginkgo biloba leaf 8:1 extract ranging from about 12.6-15.4 mg; cuscuta leaf 4:1 extract ranging from about 18-22 mg; tribulis terrestris vine 8:1 extract ranging from about 11-13.4 mg; butea superba root 8:1 extract ranging from about 15-18.3 mg; maca root 8:1 extract ranging from about 19.4-23.7 mg; pine bark extract 8:1 extract ranging from about 9.5-11.7 mg; cranberry extract ranging from about 14.4-17.6 mg; Avena sativa 8:1 extract ranging from about 1.8-2.2 mg; sarsaparilla root 8:1 extract ranging from about 1.8-2.2 mg; swedish flower pollen ranging from about 11.3-13.9 mg; pumpkin seed ranging from about 6.8-8.4 mg; di-tocopheral acetate ranging from about 1.8-2.2 mg; zinc aspartate 20% elemental ranging from about 33.8-41.3 mg; L-carnitine ranging from about 90-110 mg; L-arginine HCl ranging from about 68.4-783.6 mg; L-lysine ranging from about 54-66 mg; and Tong-Kat Ali 200:1 ranging from about 78.8-96.3 mg.

Variation 4 may include composition for sexual enhancement as in any of variations 1 through 3, wherein the composition includes about 12.4 mg saw palmetto 8:1 extract; about 12.4 mg damiana leaf 8:1 extract; about 5 mg piper nigrum fruit extract; about 14.6 mg hawthorn berry 8:1 extract; about 62 mg muira pauma bark 4:1 extract; about 15 mg Korean red ginseng 8:1 extract; about 90 mg catuaba bark 4:1 extract; about 6.6 mg epimedium sagittatum 8:1 extract; about 14 mg Ginkgo biloba leaf 8:1 extract; about 20 mg cuscata 4:1 seed extract; about 12.2 mg tribulis terrestris vine 8:1 extract; about 16.6 mg butea superba root 8:1 extract; about 21.5 mg maca root 8:1 extract; about 10.6 mg pine bark extract 8:1 extract; about 16 mg Cranberry extract; about 2 mg Avena sativa 8:1 extract; about 2 mg Sarsaparilla root 8:1 extract; about 12.6 mg Swedish flower pollen; about 7.6 mg Pumpkin seed; about 2 mg di-tocopheral acetate; about 37.5 mg zinc aspartate; about 100 mg L-carnitine; about 76 mg L-arginine HCl; about 60 mg L-lysine; and about 87.5 mg Tong-Kat Ali 200:1.

Variation 5 may include composition for sexual enhancement as in any of variations 1 through 4, wherein the composition is encapsulated within at least one of a pill or capsule.

Variation 6 may include composition for sexual enhancement as in any of variations 1 through 5, wherein the composition is administered by ingesting at least one of an oral spray or oral tincture.

Variation 7 may include composition for sexual enhancement as in any of variations 1 through 6, wherein the composition is constructed to at least one of increase anticipation prior to sexual activity, increase at least one of erectile strength or rigidity, increase pleasure or enjoyment during sexual activity including up to and prior to orgasm, increase intensity of each orgasm, increase duration of each orgasm, increase number of orgasms having a refractory period thereafter, reduce refractory periods, increase number of orgasms without a subsequent refractory period, or reduce time between orgasms.

Variation 8 may include composition for sexual enhancement as in any of variations 1 through 7, wherein the composition is encapsulated within at least one of four pills or four capsules.

In this disclosure, the descriptions of the various embodiments have been presented for purposes of illustration and are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein. Thus, the appended claims should be construed broadly, to include other variants and embodiments, which may be made by those skilled in the art.

I/We claim:

1. A composition for sexual enhancement, comprising:
saw palmetto 8:1 extract;
damiana leaf 8:1 extract;
Piper nigrum fruit extract;
hawthorn berry 8:1 extract;
muira pauma bark 4:1 extract;
Korean red ginseng 8:1 extract;
catuaba bark 4:1 extract;
epimedium sagittatum 8:1 extract;
Ginkgo biloba leaf 8:1 extract;
cuscata leaf 4:1 extract;
tribulis terrestris vine 8:1 extract;
butea superba root 8:1 extract;
maca root 8:1 extract;
pine bark extract 8:1 extract;
cranberry extract;
Avena sativa 8:1 extract;
sarsaparilla root 8:1 extract;
swedish flower pollen;
pumpkin seed;
di-tocopheral acetate;
zinc aspartate 20% elemental;
L-carnitine;
L-arginine HCl;
L-lysine; and
Tong-Kat Ali 200:1 extract.

2. A composition for sexual enhancement as in claim 1, wherein the composition is encapsulated within one or more pills or capsules.

3. A composition for sexual enhancement as in claim 1, wherein the composition comprises:
saw palmetto 8:1 extract ranging from about 11.1-13.6 mg;
damiana leaf 8:1 extract ranging from about 11.1-13.6 mg;
Piper nigrum fruit 8:1 extract ranging from about 4.5-5.5 mg;
hawthorn berry 8:1 extract ranging from about 13.1-16.5 mg;
muira pauma bark 4:1 extract ranging from about 56-68 mg;
Korean red ginseng 8:1 extract ranging from about 13.5-16.5 mg;
catuaba bark 4:1 extract ranging from about 81-99 mg;
epimedium sagittatum 8:1 extract ranging from about 5.9-7.3 mg;
Ginkgo biloba leaf 8:1 extract ranging from about 12.6-15.4 mg;
cuscuta leaf 4:1 extract ranging from about 18-22 mg;
tribulis terrestris vine 8:1 extract ranging from about 11-13.4 mg;
butea superba root 8:1 extract ranging from about 15-18.3 mg;
maca root 8:1 extract ranging from about 19.4-23.7 mg;
pine bark extract 8:1 extract ranging from about 9.5-11.7 mg;
cranberry extract ranging from about 14.4-17.6 mg;
Avena sativa 8:1 extract ranging from about 1.8-2.2 mg;
sarsaparilla root 8:1 extract ranging from about 1.8-2.2 mg;
swedish flower pollen ranging from about 11.3-13.9 mg;
pumpkin seed ranging from about 6.8-8.4 mg;
di-tocopheral acetate ranging from about 1.8-2.2 mg;
zinc aspartate 20% elemental ranging from about 33.8-41.3 mg;
L-carnitine ranging from about 90-110 mg;
L-arginine HCl ranging from about 68.4-783.6 mg;
L-lysine ranging from about 54-66 mg; and
Tong-Kat Ali 200:1 ranging from about 78.8-96.3 mg.

4. A composition for sexual enhancement as in claim 1, wherein the composition comprises:
about 12.4 mg saw palmetto 8:1 extract;
about 12.4 mg damiana leaf 8:1 extract;
about 5 mg piper nigrum fruit extract;
about 14.6 mg hawthorn berry 8:1 extract;
about 62 mg muira pauma bark 4:1 extract;
about 15 mg Korean red ginseng 8:1 extract;
about 90 mg catuaba bark 4:1 extract;
about 6.6 mg epimedium sagittatum 8:1 extract;
about 14 mg Ginkgo biloba leaf 8:1 extract;
about 20 mg cuscata 4:1 seed extract;
about 12.2 mg tribulis terrestris vine 8:1 extract;
about 16.6 mg butea superba root 8:1 extract;
about 21.5 mg maca root 8:1 extract;
about 10.6 mg pine bark extract 8:1 extract;
about 16 mg Cranberry extract;
about 2 mg Avena sativa 8:1 extract;
about 2 mg Sarsaparilla root 8:1 extract;
about 12.6 mg Swedish flower pollen;
about 7.6 mg Pumpkin seed;
about 2 mg di-tocopheral acetate;
about 37.5 mg zinc aspartate;
about 100 mg L-carnitine;
about 76 mg L-arginine HCl;
about 60 mg L-lysine; and
about 87.5 mg Tong-Kat Ali 200:1.

5. A composition for sexual enhancement as in claim 1, wherein the composition is encapsulated within at least one of a pill or capsule.

6. A composition for sexual enhancement as in claim 1 wherein the composition is administered by ingesting at least one of an oral spray or oral tincture.

7. A composition for sexual enhancement as in claim 1, wherein the composition is constructed to at least one of increase anticipation prior to sexual activity, increase at least one of erectile strength or rigidity, increase pleasure or enjoyment during sexual activity including up to and prior to orgasm, increase intensity of each orgasm, increase duration of each orgasm, increase number of orgasms having a refractory period thereafter, reduce refractory periods, increase number of orgasms without a subsequent refractory period, or reduce time between orgasms.

8. A composition for sexual enhancement as in claim 1, wherein the composition is encapsulated within at least one of four pills or four capsules.
